(19) United States
(12) Patent Application Publication
(10) Pub. No.: US 2023/0218228 A1
(43) Pub. Date: Jul. 13, 2023

(54) Noninvasive Spontaneous Respiratory Monitoring Device with Micromachined Sensing Elements

(71) Applicants: Yidong Wu, Beijing (CN); Liji Huang, Santa Clara, CA (US); Zhuo Zeng, Chengdu (CN); Hanying Wu, Nepean (CA)

(72) Inventors: Yidong Wu, Beijing (CN); Liji Huang, Santa Clara, CA (US); Zhuo Zeng, Chengdu (CN); Hanying Wu, Nepean (CA)

(73) Assignee: Wisenstech Ltd., Santa Clara, CA (US)

(21) Appl. No.: 17/573,944

(22) Filed: Jan. 12, 2022

(51) Int. Cl. A61B 5/00; A61B 5/097; A61B 5/083; A61B 5/087; A61M 16/00

(52) U.S. Cl. CPC A61B 5/682; A61B 5/6833; A61B 5/097; A61B 5/0836; A61B 5/002; A61B 5/0878; A61M 16/024; A61B 2560/0209; A61M 2230/205; A61B 2562/028

(57) ABSTRACT

The invention discloses a noninvasive spontaneous respiratory monitoring device, which comprises a sensing patch that can be placed in proximity to the nasal airway of a patient. The sensing patch measures both the flow profile and carbon dioxide concentration of a patient and wirelessly transmits the acquired data to the control circuitry for synchronizing the respiratory support of a mechanical ventilator. The device can also be used as a standalone unit for monitoring for the diagnosis purposes the spontaneous respiratory function of a patient with respiratory dysfunction.

### BACKGROUND OF THE INVENTION

### 1. Field of the Invention

This invention generally relates to medical invasive and noninvasive ventilators, and it particularly relates to a continuous measuring apparatus for the rate and direction of inspiratory & expiratory flow, and the composition of respiratory gases by a Micro Electro Mechanical systems (MEMS) sensor mounted or located on the upper lip. This invention is further related to micro-machined MEMS thermal sensing technology that can measure the continuous temperature change of respiratory gases. These data or parameters sent from MEMS sensor to the system through wireless transmission are valuable for synchronization during invasive and noninvasive ventilation (NIV) and the monitoring of spontaneous breaths during respiratory failure.

### 2. Description of the Related Art

Noninvasive ventilation would be conducted through a non-vented mask/helmet on the ICU ventilator or through a vented mask on the traditional noninvasive ventilator. Patient without spontaneous breaths is an absolute contraindication for NIV. The synchronization between the patient and the ventilator is a critical factor for the success of NIV. First of all, the synchronization relies on how quickly and accurately the ventilator detects the inspiratory initiation or expiratory cycle-off, and thus keeps pace with the spontaneous breaths.

Secondly requires a mask or nasal mask to acquire the respiratory data from the patients such that the mechanical ventilation can better serve the purpose. The typical mechanical ventilation system uses a sealing face mask to support the patient for respiration. The sealing face mask is quite limited to the cases when the patient will be immobile. The obtrusive sealing face mask has poor patient compliance and is believed to have a significant impact on the wide acceptance of the continuous positive airway pressure (CPAP) device for sleep disorders. Therefore, a more patient-friendly interface or mask is highly desired. Nasal masks on the other hand have been mostly used for oxygen therapy where oxygen is only used as supplementary assistance. The nasal mask is mobile and is less obstacle to the patient's natural respiratory passage which will be beneficial for a number of diseases such as acute respiratory disorder, or even for the pandemic cases where high flow oxygen therapy is required if it can also be applied for mechanical ventilation. Several efforts are made to develop a better nasal mask for ventilatory support. For example, Cipolloe J. et al. (U.S. Pat. No. 9,962,512, Methods, systems and devices for non-invasive ventilation including a non-sealing ventilation interface with a free space nozzle feature, May 8, 2018) disclosed a variety of nasal masks for ventilation systems that do not completely cover or seal the opening of the patient's respiratory passage. These nasal masks are non-obtrusive, more natural, and easier to adapt for mobility. Wondka A. D. disclosed a nasal mask especially to address the better flow dynamics and patient experience for CPAP (U.S. Pat. No. 7,406,966, Method and device for non-invasive ventilation with nasal interface, Aug. 5, 2008). A similar effort disclosed by Allum T. A. et al. (U.S. Pat. No. 8,677,999, Methods and devices for providing mechanical ventilation with an open airway interface, Mar. 25, 2014) also offers an open airway patient interface together with a gas delivery circuitry to optimize the performance and provide better patient experiences for the mechanical ventilation applications. However, the nasal mask brings in more variations in the respiratory data acquisition as for its nearly open space character. One of the critical control parameters for a noninvasive mechanical ventilator is the patient and ventilator synchronization, i.e., the respirator assistance action by the mechanical ventilator should be well aligned with the patient natural respiratory pattern, so the ventilator will ideally switch to the expiratory phase at the time the patient starts to breathe.

Firstly the synchronization relies on how quickly and accurately the ventilator detects the inspiratory initiation or expiratory cycle-off, and thus keeps pace with the spontaneous breaths. The existing methodologies to detect spontaneous breaths during noninvasive ventilation are a complex algorithm or mathematical model based on some indirect signals from distal flow/pressure sensors and continuous leak data. The sensors are placed away from the patient, and the delay in inhalation or exhalation patterns captured makes it difficult for the ventilator-patient to synchronize. The current NIV schemes are approximated by estimating or imitating the spontaneous inspiration & expiration other than the tidal volume and minute ventilation, which is often deviated from the actual patient respiratory data. A disclosure by Wondka A. D. et al. (U.S. Pat. No. 8,776,793, Method and devices for sensing respiration and controlling ventilator functions, Jul. 15, 2014) proposed to place a plurality of pressure sensors in the airway to capture the patient's respiratory patterns and optimize the control scheme such that a better ventilator synchronization and therapy can be achieved. The pressure sensors are, on the other hand, not a direct measurement of the patient's breath flow rate and patterns but a deductive calculation, there are still gaps in the quality of ventilator synchronization, which cannot ensure the patient experience, tolerance, compliance, and effectiveness of noninvasive ventilation. In addition, real-time respiratory monitoring is also very important for the ultimate performance of a mechanical ventilator. If the continuous and dynamic monitoring of a patient's spontaneous respiration can be precisely measured, it will help to evaluate the progress of respiratory failure before triggering any mechanical ventilation timing and strength of the ventilation treatment. Therefore, accurate and real-time measurement of the patient's respiratory pattern will be critical to improve the patient-ventilator synchronization of noninvasive ventilation, which has become an urgent technical issue for those skills in the art.

### SUMMARY OF THE INVENTION

It is therefore desired to provide the design and the making for a noninvasive spontaneous respiratory monitoring device that will be able to achieve the real-time capture with high accuracy of the patient's breath data to be used for the best control or synchronization of a mechanical ventilator. The device will further be able to directly measure the patient's flow, pressure, and other critical data such as carbon dioxide components in the open space airway or via a nasal mask configuration with minimal possibility for the removal of patient obstructiveness. The device will be in a miniaturized format and have the capability to be operated at low power with a button battery or even battery free for the best patient experience. It will also be able to have a large dynamic range and high sensitivity, and desirably with a battery free trigger if more data acquisition will be required for a large set of data acquisition.

In one object of this invention, the device provides a noninvasive spontaneous respiratory monitoring device with integrated micro-machined MEMS thermal sensing elements that can synchronize the patient's respiration with the mechanical ventilator. The device is placed in proximity of a human nasal outer passage. The device comprises a base patch that is used to fix onto the upper lip of the patient, two respiratory metering guided tubes are symmetrically arranged on the base patch. The respiratory metering guided tube is facing the nasal cavity of the patient, and each microtubule is provided with a plural of sensing elements which are used to monitor and gauge the patient's respiration and send the data to a control system for further process to achieve the optimal synchronization between the patient respiratory and the ventilation profile.

In another object of this invention, the device provides a noninvasive spontaneous respiratory monitoring device with integrated micro-machined MEMS thermal sensing elements that can synchronize the patient's respiration with the mechanical ventilator. The device is composed of a flow sensor, a carbon dioxide sensor, a wireless data transmitter, and a micro-battery. These sensing elements including the wireless data interface are integrated on a single silicon chip on which the flow sensor is made of four symmetrically arranged thermopiles that work triggered by the temperature differences due to the difference between patient respiratory temperature and the environmental temperature coupled with the cooling effects of the respiration. The design allows the flow sensor can gauge both the flow rate and the flow directions in an open space. Further, each of the thermopiles is built on a thermally isolated air cavity enabling a very fast response within 1 millisecond such that the synchronization of the metered respiratory patterns can be achieved.

In another object of this invention, the sensing elements will include a carbon dioxide sensor is made with dual and identical thermistors of which one is covered with a thermal isolation mini-cap enclosed with air while another thermistor is directly exposed to the respiratory passage. Therefore, by comparison of the thermal conductivity of these two sensors, the concentration of carbon dioxide in a patient's breath can be derived via a calibration that is performed at the time of the sensor manufacture. As the patient's respiratory composition will normally not change instantaneously, the carbon dioxide sensor can be in a pulsed pattern for which the period can be programmed as desired for the power consumption. The on-chip wireless data could be low-power Bluetooth with which the data streaming can also be programmed.

In another objective of this invention, the respiratory synchronizing device will also have a fixture for a patient to wear. The fixture can opt for further attaching and holding the nasal patch with the sensing and respiratory metering guided tube. The fixture can be directed to the head or face of the patient. This fixture can be in the form of an adhesive tape, which can be attached to the face of the patient. The fixture can also comprise a belt arranged at both ends of the patch that holds the sensing elements and the respiratory metering guided tube.

In yet another objective of this invention, the fixture can comprise two collars, which are sleeved on the auricle of the patient and attached to the head of the patient. The fixture can further comprise two mutually matched joint parts, which are looped behind the neck of the patient and connected to fix the patch onto the head of the patient.

The device provides a noninvasive spontaneous respiratory monitoring device with integrated micromachined sensing elements that can synchronize the patient's respiration with the mechanical ventilator. In an additional objective, the device can also be used as a standalone unit. In this aspect, the exhaled and inhaled airflow of the patient is gauged via the sensing elements installed inside the microtubule. Before the gauging starts, the sensing elements are in a sleep mode. The respiration airflow triggers the thermopiles that will output a current or voltage to wake up the Bluetooth data interface and the flow rate or the respiratory pattern can be streamed to the data receiver. It can also wake up the carbon dioxide sensor to meter the gas concentration in the breath and to output such via Bluetooth to the same data receiver. These data set constituent the basic information of the spontaneous respiratory function of patients. When the device is coupled to a mechanical ventilator, the data measured can assist the medical staff to adjust ventilation parameters and make mechanical ventilation consistent with or synchronize to spontaneous respiration of a patient. The device with a simple design can efficiently and accurately monitor the patient's spontaneous respiratory pattern, and significantly improve the patient-ventilator synchronization of a mechanical ventilator while improving the patient's experience in treatment, compliance, and effectiveness of noninvasive ventilation.

Other objects, features, and advantages of the present invention will become apparent to those skilled in the art through the present disclosures detailed herein wherein numerals refer to like elements.

### BRIEF DESCRIPTIONS OF THE DRAWINGS

FIG. 1 is the sketch of the key component of the noninvasive spontaneous respiratory monitoring device having sensing elements and data transmission.

FIG. 2 is a schematic drawing of the design of a micromachined integrated flow and carbon dioxide sensor chip used in the noninvasive spontaneous respiratory monitoring device.

FIG. 3 is an embodiment of the device for use of patient's spontaneous respiratory monitoring in an open space.

FIG. 4 is a schematic drawing of the use case for the noninvasive spontaneous respiratory monitoring device applied to a noninvasive ventilator.

FIG. 5 (a) is a schematic drawing of an arrangement to attach the noninvasive spontaneous respiratory monitoring device to a patient as a wearing fit.

FIG. 5 (b) is a schematic drawing of another arrangement to attach the noninvasive spontaneous respiratory monitoring device to a patient as a wearing fit.

FIG. 6 is a schematic drawing of the noninvasive spontaneous respiratory monitoring device applied for an invasive ventilator using for noninvasive monitoring purposes.

FIG. 7 is a schematic drawing of the noninvasive spontaneous respiratory monitoring device as a standalone apparatus used to monitor the patient's respiratory parameters in an open space.

FIG. 8 is a schematic drawing of the noninvasive spontaneous respiratory monitoring device for the applications in assisting sleep therapy.

### DETAILED DESCRIPTION OF THE PREFERRED EMBODIMENTS

The preferred noninvasive spontaneous respiratory monitoring device has the flow sensing elements arranged at the proximity of a patient such that the real-time respiratory pattern can be captured and timely relayed to the control circuitry of the mechanical ventilator via a wireless data streaming, that will provide the optimal real-time synchronization between the patient and the ventilator.

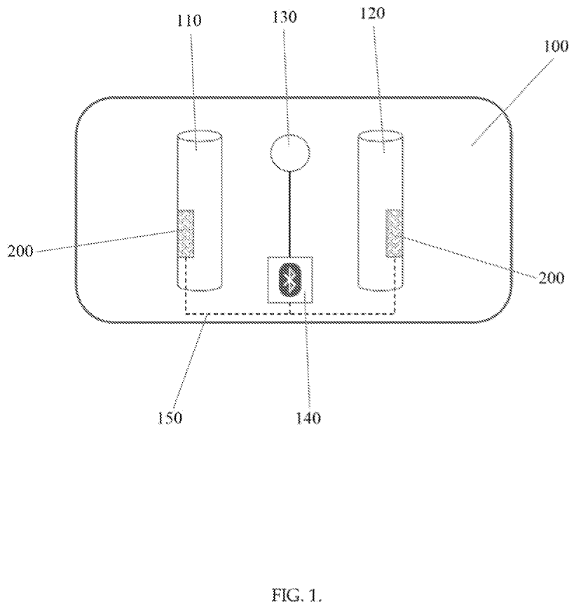

The key component of the preferred noninvasive spontaneous respiratory monitoring device is the patient's respiratory pattern capture. In the preferred embodiment, such a component will preferably be a micromachined integrated sensing chip packaged on a small patch that can be directly attached to the proximity to the patient's nasal external passage. FIG. 1 exhibited the sketch of the patch with the sensing elements 100. The patch is made of medical materials such as silica gel, flexible plastic, or fabric with one side having the composite glue that allows the patch to be fixed to the skin of a human being. Two respiratory metering guided tubes (110 and 120) are placed in parallel to each other with a distance of 20 mm that can be further finely adjusted according to a patient's actual nasal airway passage distance. The guided tubes direct and confine the flow during the patient's respiration and will significantly provide more accurate data compared to those acquired from a free space such that the desired synchronization can be better controlled. The guided tubes are preferred to be made of Nafion (polytetrafluoroethylene) with a preferred length of 15 mm, a preferred thickness of mm, and a preferred diameter of 5 mm. The Nafion tube will instantly remove the water vapor in the respiration and prevent condensation from alternating the measurement data acquisition due to the blockage of the measurement passage of the guided tube. The sensing elements or the sensor chip 200 are pre-installed inside both of the guide tubes and connected via a flexible printed circuitry 150 and the wireless data module 140. A coin battery 130 provides the power mainly to the wireless data module and for the carbon dioxide sensing elements on the sensor chip 200.

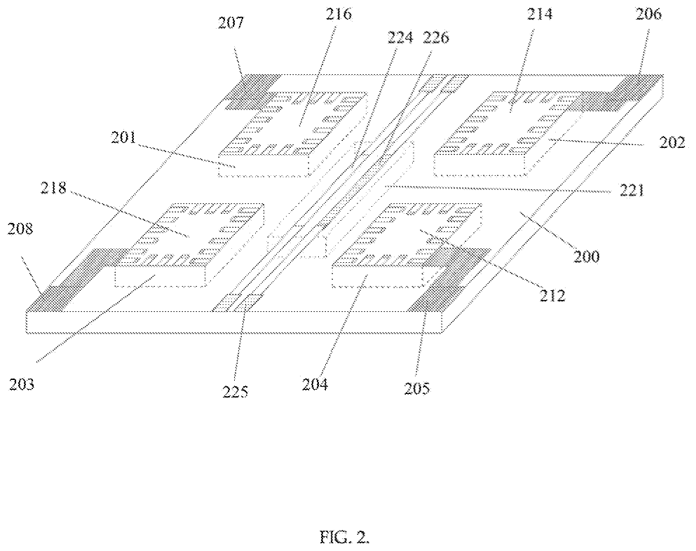

The sensing elements for both flow rate and carbon dioxide are preferred to be made on a silicon chip 200 that is exhibited in FIG. 2. In a preferred embodiment, the dimension of the sensing chip is preferred to be 2×2 mm. In order to metering the respiratory patterns in a related open space and for ultimate power reduction, the flow measurement will be preferred to utilize the thermopiles which will not require external power to operate while it can simultaneously be used as a power switch, i.e., the passive output by the thermopiles will be used to wake up the other electronics such that the components that require power supply will only be working when the respiration is present. Four thermopiles (212, 214, 216, and 218) are placed evenly at the four corners on the sensor chip surface under each is thermal isolated cavities (201, 202, 203, 204) by with micromachining process. The data acquired by these sensors are accessed via the connections and bonding pads (205, 206, 207, 208) located evenly at the four corners of the chip. Therefore, by acquiring the data from each of the flow sensors, the instant flow rate, and flow direction, as well as the flow distribution, can be obtained. In the specific implementation, the respiratory air flows via the guide tube and over the surface of the sensor chip that changes the temperature distribution at each sensor to senses the change of the temperature field at its position. The flow direction of the airflow can be determined according to the difference between each sensor, and the average value will be used to provide accurate flow data. The actual values can be obtained by calculating the reference value obtained by referring to the standard flow reference device in advance. These flow rates can be further processed to obtain the respiratory volume or efficiency, tidal volume, and minute ventilation.

The measurement of the carbon dioxide concentration in the inhaled and exhaled airway can be achieved via a pair of thermal sensors (224, 226) on the same sensor chip 200. One of the sensors (224) is covered with the passivation materials, and preferably to be silicon nitride with a thickness of 100 nm, and another sensor (226) is directly exposed to gases from the respiration. A thermal isolation cavity (221) is placed underneath to these two sensors, and the data can be accessed via the connection and the four bonding pads (225) of which one of them is marked in FIG. 2. By comparison the thermal responses and thermal conductivity of these two sensors, the carbon dioxide concentration can be determined. In the preferred embodiment, at the inspiratory phase of a normal person, the carbon dioxide concentration approximates to those in the air at the specific environment that is normally below 0.04% in volume. While at the expiratory phase, the carbon dioxide concentration would be approximately 3.8% Therefore, the carbon dioxide data acquired by the sensing elements will provide the resolution and accurate value for being used in a mechanical ventilator control to perform the inspiratory support synchronously. In addition, the level of carbon dioxide acquired at the end of the expiratory is directly associated with the ventilation level. Therefore, for the noninvasive ventilation, the ventilation would be insufficient while the carbon dioxide level at end stage of expiratory increases, it is then necessary to increase the inspiratory pressure level for the best ventilation. On the contrary, if the acquired end-expiratory carbon dioxide level decreases or stabilizes, the ventilation level could have remained unchanged.

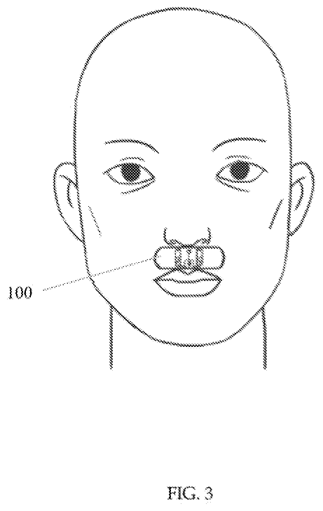

Another preferred embodiment of the noninvasive spontaneous respiratory monitoring device for a patient in open space is as exhibited in FIG. 3. The device placed on the patch (100) adheres onto the upper lip in the proximity of the external nasal airway passage of the patient. The two respiratory guided tubes will be preferably adjusted to align with the actual position of the nasal airway as close as possible. At the expiratory or inspiratory phase of the patient, the exhaled and inhaled airflow passes through the guided tube will trigger the flow sensor to output a voltage which will wake up the control electronics to start the data acquisition of the flow rate and the concentration value of carbon dioxide (or aka as carbon dioxide partial pressure) therein. The data are then wirelessly transmitted via the Bluetooth chip on the same patch (100) to the main control of the ventilator. For the best results, as there are evitable differences between the left and right nostrils and nasal septum, the averaged values of data (flow rate and carbon dioxide concentration) acquired in the two guided tubes are used for the respiratory function of patients.

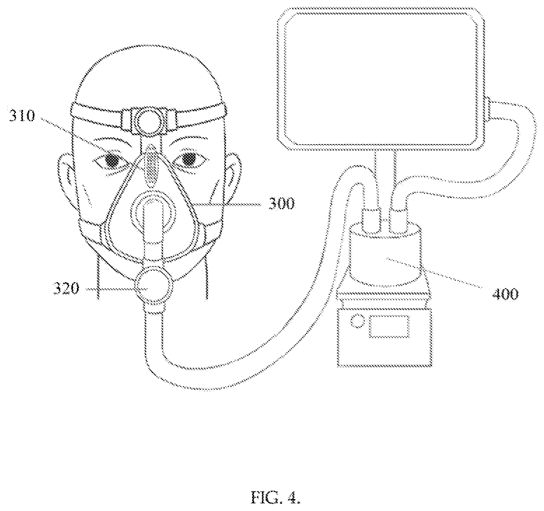

In another preferred embodiment, since the device is miniaturized, it can also be used for ventilation applications where a mask (300) is necessary. FIG. 4 exhibits such usage case. The mechanical ventilator (400) will acquire the respiratory data from the patch (100) behind the mask and adhered to the patient's nasal airway, and the data will be transmitted wirelessly to the main control of the ventilator. In this arrangement, the respiratory supports can be better synchronized with the patient's actual respiration. Traditional noninvasive ventilation is mechanical ventilation using a noninvasive bi-level ventilator (400) and through a face mask (300) with exhaust exchange (310) or exhaust valve (320). The performance for noninvasive ventilation is critically depending on the spontaneous respiration of the patient with the mask. To facilitate such setup, air leakage during the noninvasive ventilation would be necessary. The intentional air leak is normally via the exhaust exchange (310) or the exhaust valve (320) on the pipeline, but it requires the knowledge of the proper air leakage speed and exhaust volume under different expiratory pressures. Alternatively, it can also keep a constant leakage rate during the entire respiratory phase (both inspiratory and expiratory phase). In addition, since it is virtually impossible to have a complete sealing between the mask and the patient's face, some uncontrollable air leakage will exist. Although various algorithms have been applied to minimize the measurement errors due to the controlled and uncontrolled air leakage, the results are still far from satisfactory. Therefore, the data acquired directly from the patient's nasal airway will provide much accurate respiratory data compared to the existing approach where the sensors are located inside the mechanical ventilator that is adding the large uncertainties due to the air leakage stated above, as the accuracy of the respiratory data is critical for synchronizing the ventilation supports.

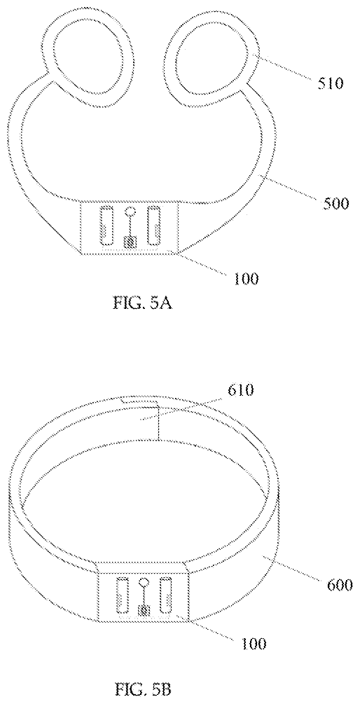

In another preferred embodiment, in addition to fixing the sensing patch (100) of the device with adhesive tape, the device also includes a wearing mechanism, which connects the patch with a soft fit onto the head or face of the patient. FIG. 5A shows the embodiment of the patch placed onto a nasal airway holder having head wearing fit (500) and collar band (510) at each end of the head wearing fit. The head-wearing fit band is sleeved on the auricle of the patient and it can also be fixed alternatively onto the head of the patient. The head-wearing fit is preferred to be made of elastic materials such as silica gel which is easy to stretch and wear for fitting to patients with variant head shapes. Furthermore, as exhibited in FIG. 5B, in another embodiment, the end of the head wearing fit can be in the form of 600 where the two ends (610) join together and can be fixed behind the neck of the patient. The end joint can be made of adhesive materials such as medical fixed tape, or preferably in the form of Velcro. It is further preferred to be made of elastic materials such as silica gel, which is easy to stretch and wear for fitting to patients with different neck shapes. The alternative wearing designs could be numerical and in various forms to fit onto a patient and have the sensing patch placed in proximity to the patient's nasal airway that can provide accurate respiratory data yet easy for a patient to adapt to optimize the ventilator synchronization as well as the improvement of the comfort, tolerance, compliance, and effectiveness of noninvasive ventilation.

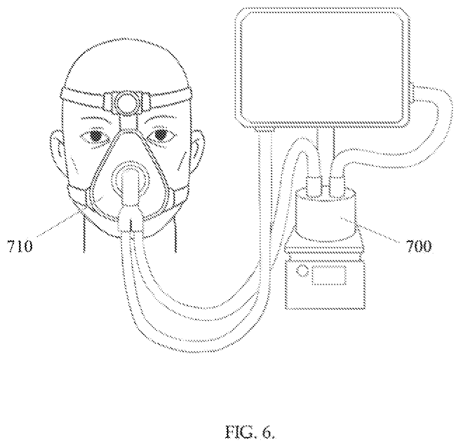

In yet another embodiment, the device is applied to noninvasive ventilation with an invasive ventilator. In this case, mechanical ventilation is achieved through a mask or helmet without a vent or exhaust valve (FIG. 6). It is used for patients with early or mild acute hypoxic respiratory failure. Because of a tight seal, ventilation synchronization is very critical yet difficult in control schemes. The current method is to acquire the flow rate and pressure data using the sensors placed inside the ventilator (700) or the pipeline to trigger and synchronize the ventilation. As such respiratory data are not directly acquired but deduced, the synchronization could sometimes be problematic. Further, most intensive care unit (ICU) invasive respirators do not have an algorithm composed of accurate mathematical and physical models to decide the inhalation and exhalation of spontaneous respiration. Adding to the fact that air leakage is inevitable, the current synchronization is far short of the clinical requirements. By applying the sensing patch to the patient's nasal airway, and acquiring the data directly to the ventilator control circuitry, the ventilator synchronization of the inhalation and exhalation of the patient can be significantly improved.

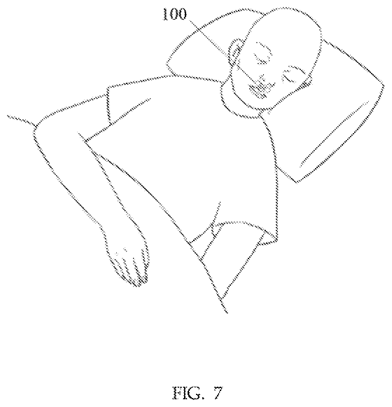

In yet another embodiment, the device is applied to patients who have or likely have respiratory failure for continuous and noninvasive monitoring of respiratory function. The current method for this task is to monitor the impedance change of the thoracic cavity through microelectrodes placed in the chest. However, this method is subject to many interference factors, such as body movement, weak respiratory impedance response, or interference from the environment. On the other hand, it can only monitor respiratory rate, and cannot provide parameters such as respiratory volume or respiratory efficiency, which has limited clinical value; it also fails to directly monitor parameters such as tidal volume, minute ventilation, exhales carbon dioxide. In most cases, respiratory function monitoring can only be performed at a hospital and not for continuous monitoring. As exhibited in FIG. 7, the device can be used as a standalone unit, and be placed at the proximity of the patient's nasal airway via the backside adhesive. The passive design of the flow sensor allows powerless data acquisition for the flow-related parameters. The device will be able to directly, continuously, and without location limitation, monitor the flow rate of inhalation and exhalation as well as the carbon dioxide concentration (partial pressure) at the nasal airway. The data can be used to calculate ventilation-related parameters, such as tidal volume and minute ventilation. The acquired end-expiratory carbon dioxide concentration directly correlates to the state of spontaneous respiratory function. For example, when the carbon dioxide concentration at the end stage of expiratory increases to above the set value, it suggests that the patient will need clinical attention. The design of ultimate low power mobile option exhibited in FIG. 7 allows continuous respiratory monitoring and data streaming. From the acquired data, the spontaneous respiratory cycle can be determined with the combination of the flow rate, flow direction, and carbon dioxide level. Parameters such as inspiratory time, expiratory time, inspiratory respiratory ratio, the respiratory frequency can then be obtained.

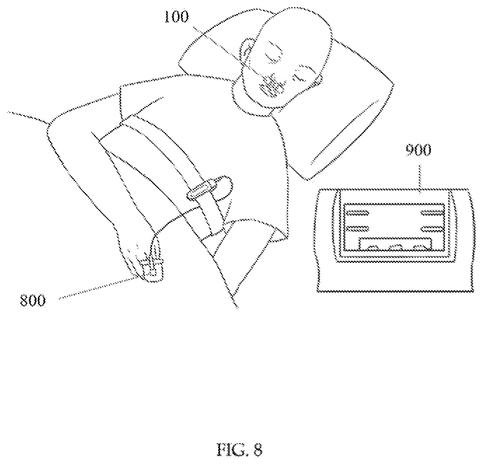

In yet another preferred embodiment, FIG. 8 exhibited another application of the device. In this embodiment, the device is applied to monitoring and assistance for sleep respiratory disorder diagnosis. The present art monitors the sleep apnea via the respiratory airflow with a nasal plug which, however, is not patient-friendly and will interfere patient's sleep position and the carbon dioxide monitor is also missing. Therefore, the embodiment of the said device can offer additional benefits for data quality. The said device directly monitors the flow rate of inspiratory and expiratory and the carbon dioxide concentration simultaneously at the nasal airway. The flow rate will disclose whether the inhalation is suspended or airflow is limited, and the end-expiratory carbon dioxide data are correlated to the state of autonomic respiratory function. These parameters will be able to determine whether the patient is suffering from insufficient ventilation or the sleep disorder is combined with ventilation insufficiency. The wireless data streaming is another advantage of the said device that will not interfere with the patient's sleep habits and can work with any sleeping position of the patient. In the preferred embodiment, the noninvasive spontaneous respiratory monitoring device can be used together with a pulse oximeter (800) that measures the percentage of blood oxygen saturation or the SpO2 levels as patients who suffer from chronic lung disease or sleep apnea and have a lower SpO2 level. The SpO2 data combined with the carbon dioxide and respiratory flow pattern can be transmitted to a nearby respiratory analyzer (900) for further data processing for the prognosis of respiratory failure, and provide an accurate clinical basis for respiratory failure treatment or intervention.

1. A noninvasive spontaneous respiratory monitoring device comprising:
one patch which can be fixed via adhesive materials or attachment fitted onto an upper lip in a proximity to a patient's nasal airway for instant monitoring a spontaneous respiratory data of the patient;
two respiratory metering guided tubes on top of the patch;
two micromachined sensing chips formed by integrating flow sensors and a carbon dioxide concentration sensor, which are located inside each of the respiratory metering guided tubes respectively; and
one low-energy Bluetooth chip for data communication.

2. The noninvasive spontaneous respiratory monitoring device of claim 1 wherein the two respiratory metering guided tubes are symmetrically arranged with a distance of 20 mm and can be finely adjusted to best match nasal cavity distances of the patient, The patch also has a 3.0 Vdc micro battery and a low-energy Bluetooth chip for power supply and data communication.

3. The noninvasive spontaneous respiratory monitoring device of claim 1 wherein the respiratory metering guided tubes are made of Nafion (polytetrafluoroethylene) materials which can effectively absorb and expel the water vapors outside the tubes, thus to avoid an adverse impact to accuracy from moisture, the respiratory metering guided tubes have a diameter of 3.0 to 6.0 mm, and length of the respiratory metering guided tubes will be ranged from 10 to 20 mm.

4. The noninvasive spontaneous respiratory monitoring device of claim 1 wherein the micromachined sensor chip is made on a silicon substrate and has a 2×2 mm square size, the micromachined sensor integrates flow and carbon dioxide concentration sensors, and both sensors are operating using thermal sensing technology.

5. The noninvasive spontaneous respiratory monitoring device of claim 1 wherein there are four flow sensors in total, each flow sensor is placed evenly at four corners of the silicon substrate, the flow sensor operates by utilizing thermopile temperature sensing technologies that is no need of power during sensing spontaneous respiration to create a temperature gradient across the sensor chip, data acquired simultaneously from the sensors at the four corners of the sensor chip will be used to calculate both flow rates and flow directions to yield final respiratory patterns of the patient.

6. The noninvasive spontaneous respiratory monitoring device of claim 1 wherein the carbon dioxide concentration sensor comprises two thermal conductivity sensing elements which are placed at central area of the silicon substrate, one of the sensing elements is covered with a thermally conductive material of silicon nitride with a thickness of 100 nm, another sensing element is directly exposed to the measurement media, by comparison of the data acquired simultaneously from both of these two elements can deduce the carbon dioxide concentration.

7. The noninvasive spontaneous respiratory monitoring device of claim 1 wherein the flow sensors will be used to generate an initial voltage or current output from sum of the four thermopiles to wake up electronics to perform a carbon dioxide concentration sensing and data streaming on the patch such that operation will be kept in a desired low power mode.

8. The noninvasive spontaneous respiratory monitoring device of claim 1 wherein a wearing fit can be used to hold the sensing patch at the proximity to the patient's nasal airway while attaching to the head or face of the patient, such a wearing fit can also be replaced with a skin-friendly adhesive tape.

9. The noninvasive spontaneous respiratory monitoring device of claim 1, is applied for open space continuous nasal airway spontaneous respiratory measurements, and measurement data are streaming to control circuitry for synchronizing the noninvasive mechanical respiratory supports.

10. The noninvasive spontaneous respiratory monitoring device of claim 1, is applied to provide a direct respiratory data to an invasive mechanical ventilator in a noninvasive control circuitry.

11. The noninvasive spontaneous respiratory monitoring device of claim 1, is applied to continuously monitor the respiratory data in a free space in proximity of patient's nasal airway to acquire sleep apnea or sleep disorder of patients for diagnosis purposes.

12. The noninvasive spontaneous respiratory monitoring device of claim 1, is coupled with an oximeter and a respiratory analyzer for applications in continuous positive airway pressure (CPAP) circumstances and provides a feedback data for automatic control circuitry of a CPAP ventilator.
